(19) United States
(12) Patent Application Publication
(10) Pub. No.: US 2026/0248583 A1
(43) Pub. Date: Aug. 27, 2026

(54) SYSTEM FOR DELIVERY OF A FIDUCIAL MARKER

(71) Applicant: Teleflex Life Sciences LLC, Wilmington, DE (US)

(72) Inventors: Jacqueline Nerney Welch, Moraga, CA (US); Floria Cheng, San Francisco, CA (US); Jolene Cutts, San Francisco, CA (US); Alexander Charles Gordon, San Carlos, CA (US); Kevin Alexander Lessard, San Francisco, CA (US); Curtis Yarra, Oakland, CA (US); Christopher Zaler, Los Gatos, CA (US); Joseph Catanese, Oakland, CA (US)

(21) Appl. No.: 19/333,142

(22) Filed: Sep. 18, 2025

Related U.S. Application Data
(63) Continuation of application No. 17/283,497, filed on Oct. 30, 2019, now Pat. No. 12,440,301.
(60) Provisional application No. 62/756,907, filed on Nov. 7, 2018.

(51) Int. Cl. A61B 90/00; A61B 17/00; A61B 17/04

(52) U.S. Cl. CPC A61B 90/39; A61B 17/00234; A61B 2017/00004; A61B 2017/00274; A61B 2017/0419; A61B 2017/0464; A61B 2090/3908; A61B 2090/3966; A61B 2090/3987

(57) ABSTRACT

A transurethral system for delivering and depositing fiducial markers usable to delineate the prostate during radiation therapy.

### CROSS REFERENCE TO RELATED APPLICATIONS

This application is a continuation of U.S. application Ser. No. 17/283,497, entitled “SYSTEM FOR DELIVERY OF A FIDUCIAL MARKER” and filed Apr. 7, 2021, which in turn is a U.S. national stage application under 35 U.S.C. 371 of PCT Application No. PCT/US2019/058708, entitled “SYSTEM FOR DELIVERY OF A FIDUCIAL MARKER” and filed Oct. 30, 2019, which claims priority to U.S. provisional patent application no. 62/756,907, entitled “DEVICE AND SYSTEM FOR DELIVERY OF FIDUCIAL MARKERS” and filed on Nov. 7, 2018, each of which is herein incorporated by reference in its entirety.

### BACKGROUND OF THE INVENTION

The present invention relates generally to medical devices, and more particularly to systems for delivering fiducial markers to an interventional site within the body of a human or animal subject.

Prostate cancer is the second most common cancer, as well as the second leading cause of cancer-related deaths, among men in the United States. Presently, one in nine men in the United States is diagnosed with prostate cancer in their lifetime. The American Cancer Society estimates about 165,000 new cases of prostate cancer and 29,000 deaths from prostate cancer will occur in 2018.

External Beam Radiation Therapy (EBRT) is a core treatment modality for patients with non-metastatic prostate cancer and is used for nearly one third of all patients receiving prostate cancer therapy.

Three-dimensional (3D) conformal radiation therapy (3DCRT) is an individualized, image-guided EBRT technique whereby a radiation dose is planned and delivered such that the high-dose volume is restricted to a predetermined target volume. 3DCRT utilizes 3D digital data sets representative of patient tumors and adjacent anatomy. These data sets are then used to select the number, direction, and arrangement of radiation beams to deliver the high-dose radiation to a volume of tissue. This targeting process allows higher doses of radiation to be delivered to cancer cells while reducing the amount of off-target radiation received by adjacent, healthy tissue. Accordingly, effective delivery of conformal radiation therapy (RT) relies on accurately locating the prostate gland and its boundaries.

Fiducial markers, small radiopaque objects typically made from gold or platinum, can be implanted in the prostate gland prior to RT and subsequently used to localize the prostate gland and associated tumor and to accurately deliver the radiation dose during treatment. Fiducial markers known in the prior art are designed to be biologically inert. Fiducial markers are typically delivered using a needle applicator, whose length can range from 20 cm to 30 cm. Ideally, fiducial markers are implanted 3 mm to 5 mm from the edge of the prostate gland, which is covered by a tissue capsule.

Fiducial markers are typically implanted using a transrectal or transperineal approach. In both approaches, the fiducials are implanted from a few days to a week before the initial scans that are then used to plan the radiation treatment. The transrectal procedure carries with it a risk of rectal bleeding and both procedures carry the risk of infection, and of urosepsis in particular. Other complications of fiducial marker placement include pain, fever, voiding issues, hematuria, hematospermia, inflammatory bowel disease, and complications related to migration of the markers.

Patients with prostate cancer frequently experience lower urinary tract symptoms (LUTS) including urinary hesitancy, chronic urinary retention, and nocturia. Furthermore, patients undergoing EBRT are more likely to experience LUTS, particularly if the patient also suffers from Benign Prostatic Hyperplasia (BPH), a condition characterized by noncancerous enlargement of the prostate.

BPH can be treated by a variety of surgical and nonsurgical procedures including a minimally invasive transurethral procedure that implants small prostatic anchors to hold the prostate lobes in compression and relieve blockage of the urethra.

Such a system could also be used to deliver fiducial markers for EBRT as the placement of the anchors can be used to delineate the prostate during the planning and execution of RT. In this way, fiducial markers could be implanted without the need for an additional procedure and/or in a way that identifies the boundary of the prostate. Embodiments of the invention disclosed herein address various aspects of such as system and the methods of use of that system.

### SUMMARY OF THE INVENTION

Embodiments of the present invention are directed towards a system for transurethral delivery of fiducial markers to the prostate gland that can be used to visualize and target the prostate during radiation therapy. One embodiment of the system includes a device having at least one fiducial marker, an anchor assembly with a first and second anchoring component and a connector, and a delivery device.

The fiducial markers can be made of materials that are detectable by suitable medical imaging equipment. The fiducial markers can be formed integrally with or are attached to various portions of the anchor assembly by swaging, molding, plating, or embedding during formation of the anchor assembly.

In some embodiments, the fiducial marker is a dye or contrast agent deposited on the connector or delivered by a needle advanceable from the fiducial delivery device. The needle can be used to inject or release the dye or contrast agent into the prostatic lobes or extra-prostatic space.

Various embodiments of the anchor assembly with fiducial markers are disclosed and described herein. Moreover, various ways in which fiducial markers can be deposited to an interventional site by the fiducial delivery device independent of the anchor assembly are discussed.

Other features and advantages of embodiments of the present invention will become apparent from the following description, taken in conjunction with the accompanying drawings, which illustrate, by way of example, certain principles of the invention.

### BRIEF DESCRIPTION OF THE DRAWINGS

FIG. 1 is a top view of an assembled anchor assembly.

FIG. 2 is a cross-sectional view through the prostatic urethra with the anchor assembly applied to the prostate.

FIG. 3A is a top view of one embodiment of an anchor assembly with fiducial marker(s).

FIG. 3B is an enlarged, top view of one embodiment of an anchor assembly with fiducial marker(s).

FIG. 3C is an enlarged, top view of one embodiment of a fiducial marker.

FIG. 4A is a top view of another embodiment of an anchor assembly with fiducial marker(s).

FIG. 4B is an enlarged, top view of another embodiment of an anchor assembly with fiducial marker(s).

FIG. 4C is an enlarged, top view of another illustrative embodiment of an anchoring component with fiducial marker(s).

FIG. 5 is a top view of an embodiment of an anchor assembly with fiducial marker(s).

FIG. 6 is a top view of an embodiment of an anchor assembly with fiducial marker(s).

FIG. 7A is a top view of an embodiment of an anchor assembly with a fiducial tag.

FIG. 7B is an enlarged, top view of an embodiment of an anchor assembly with a fiducial tag.

FIG. 8A is a top view of another illustrative embodiment of a connector with a fiducial marker.

FIG. 8B is a top view of another illustrative embodiment of an anchoring component with a fiducial marker.

FIG. 9 is a top view of an illustrative embodiment of an anchor assembly with a radiopaque wire.

FIG. 10A is a top view of another illustrative embodiment of an anchoring component with a fiducial marker.

FIG. 10B is a top view of still another illustrative embodiment of an anchoring component with a fiducial marker.

### DETAILED DESCRIPTION OF ILLUSTRATIVE EMBODIMENTS

The present disclosure is directed to a system for transurethral delivery of fiducial markers to the prostate that can be used to visualize and target the prostate during radiation therapy. The figures herein are provided by way of example and not limitation, and the description of the various embodiments of the invention includes all aspects of the figures.

Generally, embodiments of the system according to the present disclosure include an anchor assembly that modifies, contours, or otherwise manipulates prostatic tissue and includes material or one or more features detectable by medical targeting and/or imaging modalities. Such modalities presently include, but are not limited to, ultrasound, magnetic resonance imaging (MRI), and radiography (such as x-ray imaging and including computed tomography). For radiography, the suitable materials are radiopaque and include, but are not limited to, gold, platinum, or tantalum. To the extent an embodiment refers to a fiducial marker as being radiopaque, it should be understood that fiducial markers having properties detectable by other medical targeting and/or imaging modalities are also included in such an embodiment. Further, the shape, size, and surface texture (including roughness) of an anchor assemble can be altered improve the ability for targeting and/or imaging modalities to detect the anchor assembly.

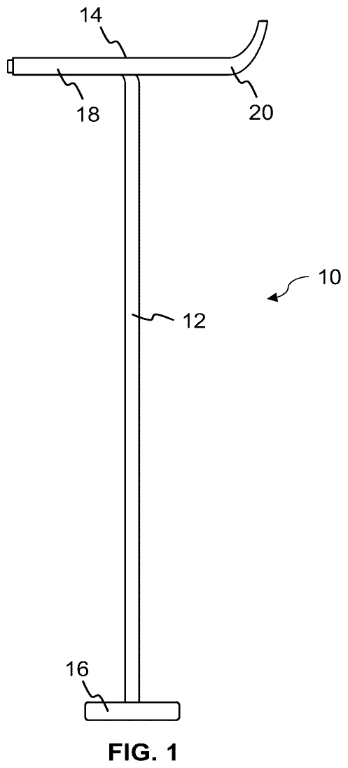

FIG. 1 is a top view of an assembled anchor assembly. As shown in FIG. 1, implant or anchor assembly 10 includes connector 12 situated between first anchoring component 14 and second anchoring component 16. Connector 12 can be formed from suitable material that provides a desired holding force between the first and second anchoring components. In some embodiments, connector 12 can be formed from conventional suture material. In preferred embodiments, connector 12 is monofilament polyethylene terephthalate (PET). The flexible material of connector 12 can accommodate access and placement of anchor assembly 10 to various positions in the prostate and transmit necessary forces between the first and second anchoring components. First anchoring component 14 can be attached to connector 12 by various means including, but not limited to, adhesive bonding or thermal bonding or can include tabs or other structures capable of forming a locking arrangement with connector 12. Alternatively, first anchoring component 14 can be crimped or secured directly to connector 12. In some embodiments, connector 12 includes a structure(s) which is complementary to first anchoring component 14 to facilitate attachment.

Similar approaches can be used to secure connector 12 to second anchoring component 16. However, in preferred embodiments, second anchoring component is secured to connecter 12 during the time of placement of anchor assembly 10 within a patient. That is, in preferred embodiments, first anchoring component 14 can be attached to connector 12 during the manufacturing process, and second anchoring component is left unsecured to connecter 12. An unassembled anchor assembly is loaded into a delivery system. First anchoring component 14 and attached connector 12 are delivered to a location within or near the prostate gland. Second anchoring component 16 is then secured to connector 12 by the delivery system within or near the prostate gland.

Various embodiments of an anchor assembly or selected portions thereof and an anchor assembly delivery device are detailed or contemplated in U.S. Pat. No. 7,896,891 entitled “Apparatus and Method for Manipulating or Retracting Tissue and Anatomical Structure” and U.S. Pat. No. 7,914,542 entitled “Devices, Systems and Methods for Treating Benign Prostatic Hyperplasia and Other Conditions” which are hereby incorporated by reference in their entireties.

First anchoring component 14 and second anchoring component 16 can be formed from conventional biocompatible materials such as, but not limited to, stainless steel and nitinol. First anchoring component 14 includes a first portion 18 and a second portion 20. In some preferred embodiments, first portion 18 is a generally cylindrical structure while second portion 20 is at least partially cylindrical and includes a curved, angled, or otherwise bent structure or leg.

The lumen within the generally cylindrical portion of first anchoring component 14 can facilitate attachment of connector 12 by placing connector 12 coaxially within the lumen of the generally cylindrical portion of first anchoring component 14. Once connector 12 is within the lumen of the generally cylindrical portion of first anchoring component 14, connector 12 can be secured via various means including, but not limited to, adhesive bonding or thermal bonding. First anchoring component 14 can include tabs or other structures capable of forming a locking arrangement with connector 12.

In one preferred embodiment, the process for attaching first anchoring component 14 and connector 12 can include creating a fiducial marker at or near the junction of first anchoring component 14 and connector 12. In the case of bonding connector 12 to first anchoring component 14, the bonding process can introduce a fiducial marker material. For example, a radiopaque adhesive material can be used to bond first anchoring component 14 and connector 12. A radiopaque foil, sheet, or other thin structure can be used to form an interference fit between first anchoring component 14 and connector 12 by sandwiching the foil between first anchoring component 14 and connector 12. A radiopaque cap, plug, or similar structure can be used to anchor the end of connector 12 to first anchoring component.

Still further, a radiopaque material can be introduced during a thermal bonding step to bond first anchoring component 14 and connector 12. For example, when connector 12 is within the lumen of the generally cylindrical portion of first anchoring component 14, a fiducial marker can be embedded at or near the joint between first anchoring component 14 and connector 12 by heating the joint and introducing the fiducial marker. The heating step can be used form a cap on a portion of connector 12 that extends outside the the lumen of the generally cylindrical portion of first anchoring component 14, thereby forming a secure connection between first anchoring component 14 and connector 12. This cap could contain a fiducial marker.

While one embodiment of anchor assembly 10 is illustrated in FIG. 1 with a single first anchoring component 14, in other embodiments, an anchor assembly can include a plurality of first anchoring components spaced to suit a particular application. When a plurality of first anchoring components are included, the first anchoring components can be spaced at time of deployment, or can be spaced after deployment with subsequent manipulation. The first anchoring components can be provided at fixed, spaced positions, or can be provided such that the first anchoring components are able to be moved into the desired spaced arrangement.

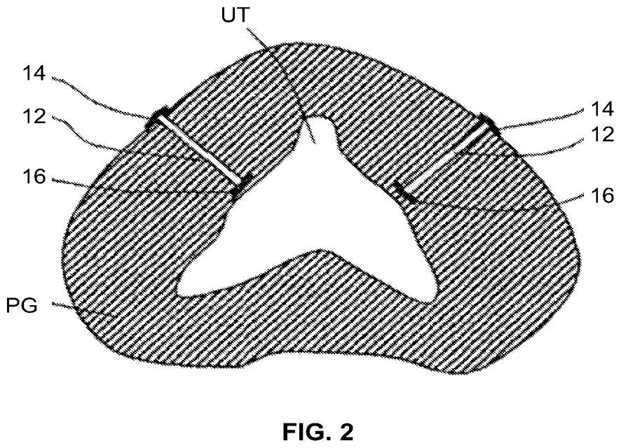

FIG. 2 is a cross-sectional view through the prostatic urethra (the portion of the urethra that passes through the prostate gland) with the anchor assembly applied to the prostate. As shown in FIG. 2, an anchor assembly can be employed to manipulate the prostate gland (PG) by positioning first anchoring component 14 at the outer surface of the prostatic capsule and second anchoring component 16 at or near the inner surface of the urethra (UT) such that the tissue captured between the anchors is held in a compressed state by tensioned connector 12. While a single anchor assembly is shown on each lateral lobe of FIG. 2, it will be understood that multiple anchor assemblies can be applied to each lateral lobe depending on the application. The length of the connector element can also be customized for various applications.

In preferred embodiments, the anchor assembly is part of a transurethral system that localizes fiducial markers to the prostate for the purpose of marking and/or delineating the prostate, or a portion thereof, during x-ray imaging, MRI, CT scan, and/or radiotherapy. This can be achieved by incorporating or connecting plates, bands, or seeds of markers made of inert, radiopaque material such as, but not limited to, gold, platinum, or tantalum to various portions of the anchor assembly during manufacture or formation of the anchor assembly.

Markers can be various shapes and sizes. In some preferred embodiments, markers are spherical or cylindrical in shape with a diameter in the range of 0.5-1.5 mm and a length in the range of 2-5 mm that are easily identified during targeting and/or imaging.

In some embodiments, radiopaque markers (subsequently referred to as “markers”) are incorporated by swaging, molding, plating, or embedding radiopaque material onto a portion of the anchor assembly. In preferred embodiments, at least three markers are used during the procedure to establish the required number of fiducial targets. More than one marker can be present on an anchor component, connector, or anchor assembly. To the extent the markers are included on the connector, the number of markers should be such that the flexibility of the connector is maintained.

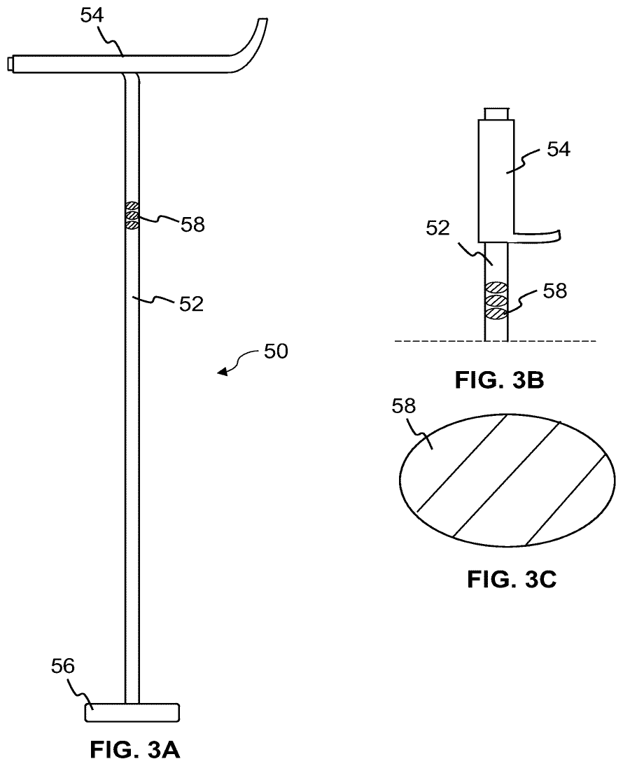

FIG. 3A is a top view of one embodiment of an anchor assembly with fiducial marker(s). FIG. 3B is an enlarged, top view of one embodiment of an anchor assembly with fiducial marker(s). As shown in FIGS. 3A-3B, anchor assembly 50 includes connector 52 with markers 58. Markers can be clustered together and biased toward the end of connector 52 that contacts first anchoring component 54 or second anchoring component 56. Alternatively, markers 58 can be spaced at equidistant intervals along the length of connector 52.

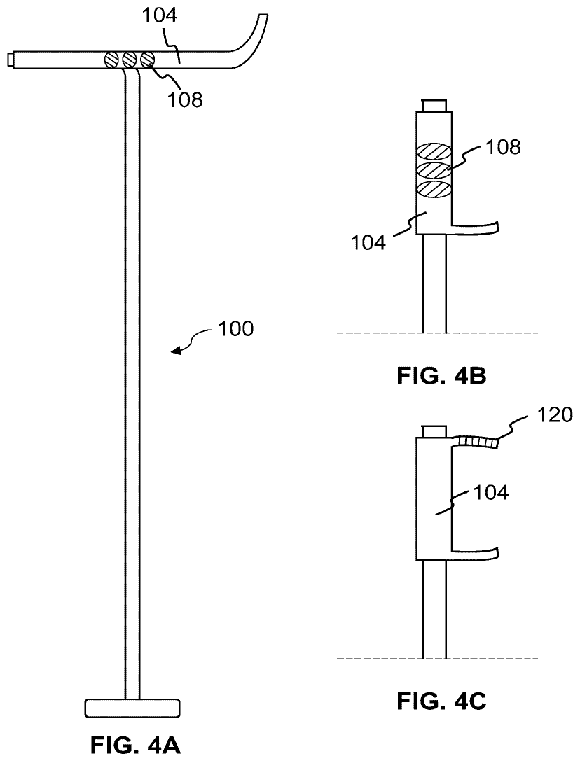

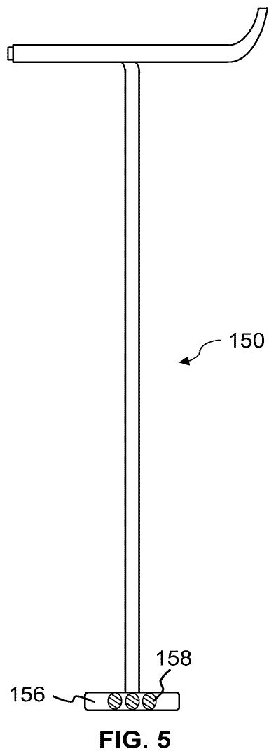

In other embodiments, radiopaque markers can be included in the anchoring portions of the anchor assembly. FIG. 4A is a top view of another embodiment of an anchor assembly with fiducial marker(s). FIG. 4B is an enlarged, top view of another embodiment of an anchor assembly with fiducial marker(s). FIG. 4C is an enlarged, top view of another illustrative embodiment of an anchoring component with fiducial marker(s). FIGS. 4A and 4B illustrate anchor assembly 100 with a plurality of markers 108 incorporated in first anchoring component 104. Alternatively, as shown in FIG. 4C, anchor assembly 100 can include an anchoring component 104 with a second radiopaque leg 120 in lieu of or in addition to markers 108. FIG. 5 is a top view of an embodiment of an anchor assembly with fiducial marker(s). FIG. 5 illustrates anchor assembly 150 with a plurality of markers 158 in second anchoring component 156. It is also contemplated that the first and second anchoring components or portions thereof can be manufactured from radiopaque material or include coils, anchors, or other suitable structures that confer the ability of the anchor assembly to manipulate the prostate and function as fiducial markers.

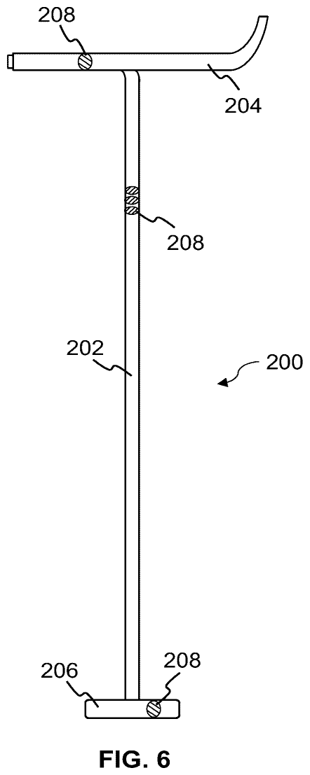

FIG. 6 is a top view of an embodiment of an anchor assembly with fiducial marker(s). FIG. 6 shows an embodiment of anchor assembly 200 in which markers 208 are included in first anchoring component 204, connector 202, and/or second anchoring component 206. Use of markers 208 on the first and/or second anchoring components, in addition to the markers on connector 202, can allow for additional positional information of the prostate to be obtained and further refine the boundaries of the prostate when during imaging and/or radiotherapy.

In some embodiments, rather than using distinct fiducial markers, the connector or suture can be coated, filled, or saturated with radiopaque dye or contrast agent such as barium sulfate. It is contemplated that the anchoring components of the anchor assembly can also be configured with reservoirs filled with radiopaque dye or contrast agent.

Alternatively, co-delivery of the radiopaque dye or contrast agent could be achieved by including an internal reservoir filled with radiopaque dye or contrast agent housed in the delivery device such that when the anchor assembly is advanced from the device, it picks up a desired quantity of the dye or agent.

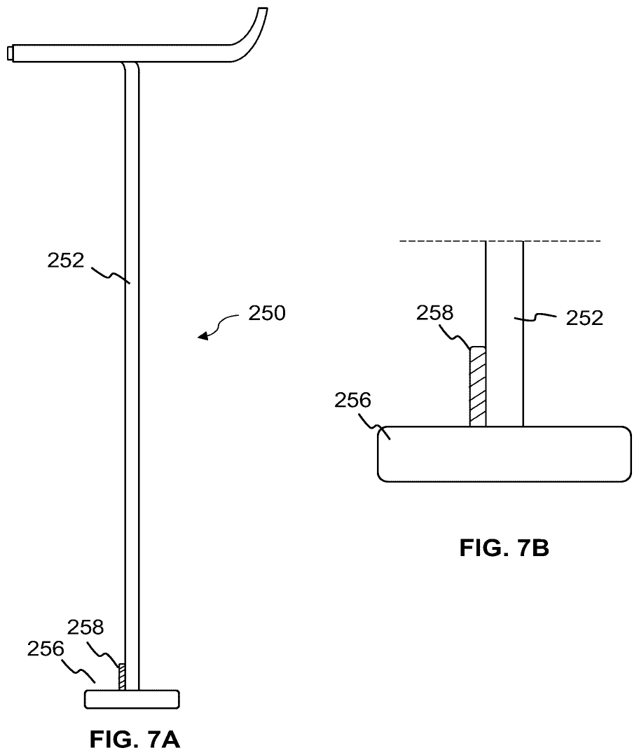

FIG. 7A is a top view of an embodiment of an anchor assembly with a fiducial tag. FIG. 7B is an enlarged, top view of an embodiment of an anchor assembly with a fiducial tag. In FIGS. 7A nd 7B, rather than swaging a radiopaque marker onto a component of the anchor assembly, an embodiment with a separate radiopaque tag is shown. Here, radiopaque tag 258 is a thin sheet of radiopaque material situated between second anchoring component 256 and connector 252. Tag 258 can be attached to second anchoring component 256 and connector 252 by conventional means.

In embodiments of the anchor assembly in which a plurality of markers are used, each marker can have the same dimensions and thickness or be of varying dimensions and thicknesses. Varying the thickness of radiopaque markers can provide directionality or orientation-related information of the prostate.

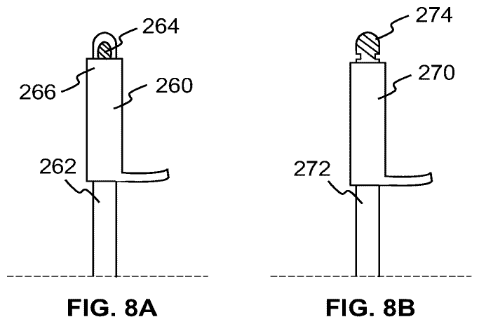

Alternative embodiments of anchor assemblies incorporating radiopaque material are shown in FIGS. 8A and 8B. FIG. 8A is a top view of another illustrative embodiment of a connector with a fiducial marker. FIG. 8B is a top view of another illustrative embodiment of an anchoring component with a fiducial marker. FIG. 8A shows connector 262 in which radiopaque marker 264 has been incorporated into a rounded tip of the connector that extends beyond cylindrical end 266 of first anchoring component 260. This allows marker 264 to be situated in the extra-prostatic space when the anchor assembly is implanted and prevents or minimizes the creation of imaging artifacts. Markers may be placed in the intra-prostatic space in order to localize specific features within the prostate. FIG. 8B shows a partial view of an anchor assembly that includes bullet tip 274 made of radiopaque material. Tip 274 can be secured to the end of first anchoring component 270 or configured to be continuous with connector 272.

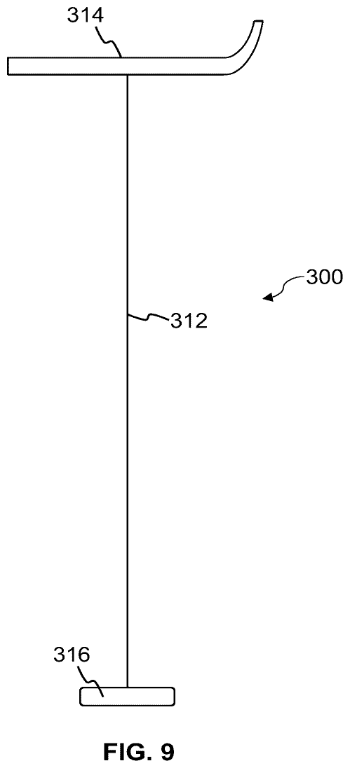

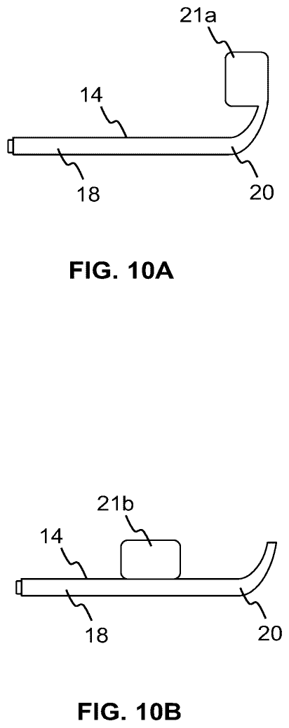

FIG. 9 is a top view of an illustrative embodiment of an anchor assembly with a radiopaque wire. Another embodiment of an anchor assembly is shown in FIG. 9. Anchor assembly 300 includes first anchoring component 314, second anchoring component 316, and connector 312. In this embodiment, connector 312 is a wire made from or coated with suitable radiopaque material such as gold.

Application of the anchor assembly to the prostate can also impart information on prostate health. For example, the system can be configured to supply data on tissue stiffness or other characteristics during implantation of the anchor assembly. Such data could be used to distinguish normal, healthy tissue from abnormal tissue. After implantation, movement or other behavior of the markers over time may provide further information on prostate health, including, but not limited to, changes in size.

Fiducial markers can also be delivered to the prostate by the anchor assembly delivery device independent of the anchor assembly. Various embodiments of an anchor delivery system are detailed or contemplated in U.S. Pat. No. 9,504,461 entitled “Anchor Delivery System” which is hereby incorporated by reference in its entirety. The delivery device includes various subassemblies configured to deploy one or more anchor assemblies and/or deliver therapeutic or diagnostic agents to an interventional site within the body of a patient. In one aspect, the delivery device can include a cartridge carrying the anchor assembly or therapeutic/diagnostic agent and a handle assembly configured to couple with the cartridge such that mechanical energy loaded in at least one spring mechanism within the handle is transferred to the cartridge to deploy the anchor assembly or agent. The device includes an actuator configured to initiate transfer of the mechanical energy to the spring mechanisms.

The delivery device can also include an elongate member connected to a handle assembly that can be inserted into the urethra of a patient and advanced to establish contact with the prostate gland. The elongate member can house components to construct an anchor assembly. The elongate member can also house tools controllable by an actuator and advanceable from the elongate member such as a needle or other penetrating member that deploy one or more anchor assemblies and/or deliver therapeutic or diagnostic agents to the prostate.

It is contemplated that the hollow needle or penetrating member can be used to deliver radiopaque dye or contrast agent to the extra-prostatic space or to inject dye or agent into the prostatic lobes or urethral tissue for radiotherapy or imaging. Alternatively, the needle exit point can include tape or other adhesives configured to deposit radiopaque material onto the needle, anchor assembly, implant, or other penetrating members when the tape is pierced or otherwise contacted.

The delivery device can also be used to deliver and deposit radiopaque or therapeutic seeds, coils, or anchors into the tissue of the prostate, urethra or bladder. In some embodiments, the delivery device includes a fiducial cartridge, separate from the anchor assembly-delivering cartridge that delivers the fiducial marker as part of the actuator deployment sequence.

The delivery device can also be used to modify an existing or previously implanted anchor assembly by affixing radiopaque seeds or other radiopaque material to the anchoring components and/or connector.

In each of the detailed and contemplated embodiments, the fiducial markers can be used to delineate the prostate for, at least, the planning and execution of radiation therapy to treat prostate cancer or other cancers. The number and placement of fiducial markers can be optimized to allow triangulation and measurement of anatomical position in different planes. For procedures in which multiple anchor assemblies are used to manipulate each of the lateral prostatic lobes, fiducial markers need not be incorporated into every anchor assembly. Fiducial markers can be used to identify tissue planes or other tissue features within an organ, gland, or other collection of tissue.

In a typical procedure, the physician places implants in the anterior portion of the prostate. The physician does not place implants in the posterior aspect of the prostate because implants positioned in the posterior aspect could compress the neurovascular bundles, which are located at nearby. Similarly, placing implants in the trust your aspect of the prostate could result in damage to the rectum. However, for use as a fiducial marker, implants can be deployed in the posterior aspect of the prostate. In such an embodiment, only the distal section of the anchor assembly would be implanted. With only the distal section implanted, there would be no compression on the neurovascular bundles because the proximal anchor would not be placed, and it is this anchor that typically facilitates holding compression on tissue. Thus, it is possible to delineate the outer capsule of the prostate gland in the posterior aspect, which is often where cancerous tissue is located.

An exemplary embodiment of the invention disclosed herein is a system for delivering and depositing a fiducial marker to tissue at an interventional site within the body of a human patient, where the system includes: a first fiducial marker made of radiopaque material; at least two anchor assemblies, the anchor assemblies including a first anchoring component, a second anchoring component, and a connector; and a fiducial delivery device, the fiducial delivery device carrying the anchor assembly and comprising an actuator usable to deploy the anchor assembly and an elongate member to be inserted and advanced to the interventional site.

The exemplary embodiment includes an aspect wherein the fiducial marker is attached to a portion of each anchor assembly by swaging, molding, plating, or embedding during formation of the anchor assembly.

The exemplary embodiment includes an aspect wherein the first fiducial marker is attached to the first anchoring component.

The exemplary embodiment includes an aspect wherein the system includes a second fiducial marker and a third fiducial marker wherein the second and third fiducial markers are made of radiopaque material and attached to the first anchoring component.

The exemplary embodiment includes an aspect wherein the first fiducial marker is attached to the connector and wherein the second and third fiducial markers are made of radiopaque material and attached to the connector.

The exemplary embodiment includes an aspect wherein the first fiducial marker is attached to the second anchoring component and wherein the second and third fiducial markers are made of radiopaque material and attached to the second anchoring component.

The exemplary embodiment includes an aspect wherein the radiopaque material of the first fiducial marker is a dye or contrast agent. The exemplary embodiment includes an aspect wherein the dye or contrast agent coats the length of the connector. The exemplary embodiment includes an aspect wherein the first anchoring component and/or the second anchoring component further comprises a reservoir to hold the dye or contrast agent.

The exemplary embodiment includes an aspect wherein the first fiducial marker is made of metal. The exemplary embodiment includes an aspect wherein the first fiducial marker is made of gold, platinum, or tantalum. The exemplary embodiment includes an aspect wherein the first fiducial marker is a band or a seed.

The exemplary embodiment includes an aspect wherein first anchoring component, the second anchoring component, the connector, or a portion thereof is made of radiopaque material and serves as the first fiducial marker.

The exemplary embodiment includes an aspect wherein first anchoring component, the second anchoring component, the connector, or a portion includes features that increase radiopacity.

The exemplary embodiment includes an aspect wherein the first fiducial marker is a tag made of radiopaque material situated between the second anchoring component and the connector.

The exemplary embodiment includes an aspect wherein the system includes a plurality of fiducial markers wherein the plurality of fiducial markers are made of metal. The exemplary embodiment includes an aspect wherein the plurality of fiducial markers are made of gold, platinum, or tantalum.

The exemplary embodiment includes an aspect wherein the first anchoring component comprises a leg made of radiopaque material that serves as the first fiducial marker.

The exemplary embodiment includes an aspect wherein the connector includes a rounded tip that extends into the extra-prostatic space when the anchor assembly is implanted into a prostatic lobe of a patient, wherein the rounded tip includes radiopaque material and serves as the first fiducial marker.

The exemplary embodiment includes an aspect wherein the fiducial delivery device further comprises a needle advanceable from the elongate member.

The exemplary embodiment includes an aspect wherein the fiducial marker is manipulated after being delivered.

The exemplary embodiment includes an aspect wherein the fiducial marker is placed in intra-prostatic space, extra-prostatic space, on the prostatic capsule, or combinations thereof.

The exemplary embodiment includes an aspect wherein the fiducial marker is biodegradable.

The exemplary embodiment includes an aspect wherein the fiducial marker delivers therapy to tissue.

The exemplary embodiment includes an aspect wherein the needle is configured to deliver the first fiducial marker. The exemplary embodiment includes an aspect wherein the system includes a second fiducial marker and a third fiducial marker wherein the second and third fiducial markers are delivered to the interventional site by the needle. The exemplary embodiment includes an aspect wherein the first fiducial marker is a dye or contrast agent delivered by the needle. The exemplary embodiment includes an aspect wherein the needle injects the dye or contrast agent into tissue adjacent to the prostate. The exemplary embodiment includes an aspect wherein the needle injects the dye or contrast agent into the prostatic lobes. The exemplary embodiment includes an aspect wherein the needle injects the dye or contrast agent into the extra-prostatic space.

The exemplary embodiment includes an aspect wherein at least two of the first, second, or third fiducial marker span a gland or organ. The exemplary embodiment includes an aspect wherein the fiducial marker is placed at a tissue plane.

The exemplary embodiment includes an aspect wherein the system delivers fiducial markers to the prostate for the purpose of delineating the prostate during radiotherapy.

While particular elements, embodiments and applications of the present invention have been shown and described, it will be understood that the invention is not limited thereto since modifications can be made by those skilled in the art without departing from the scope of the present disclosure, particularly in light of the foregoing teachings.

1. A system for delivering and depositing at least one fiducial marker to tissue at an interventional site within a body of a patient, comprising:
an anchor assembly deployable at the interventional site, the anchor assembly comprising a first anchoring component, a second anchoring component, a connector disposed between the first anchoring component and the second anchoring component, and the at least one fiducial marker, wherein:
the at least one fiducial marker is made of a radiopaque material incorporated onto at least a portion of the first anchoring component, the second anchoring component, the connector, or a combination thereof, and
the anchor assembly is configured to hold tissue captured between the first anchoring component and the second anchoring component in a compressed state via the connector such that the at least one fiducial marker delineates a boundary of the tissue; and
a delivery device carrying the anchor assembly, the delivery device comprising:
an elongate member configured to be inserted into the body of the patient and advanced to the interventional site; and
an actuator usable to deploy the anchor assembly at the interventional site.

2. The system of claim 1, wherein the at least one fiducial marker is attached to the first anchoring component by swaging, molding, plating, or embedding during formation of the anchor assembly.

3. The system of claim 1, wherein the at least one fiducial marker is attached to the connector.

4. The system of claim 3, wherein the at least one fiducial marker comprises three fiducial markers attached to the connector.

5. The system of claim 1, wherein the at least one fiducial marker is attached to the second anchoring component.

6. The system of claim 1, wherein the first anchoring component comprises a leg made of the radiopaque material that serves as the at least one fiducial marker.

7. The system of claim 1, wherein the connector includes a rounded tip configured to extend beyond the first anchoring component when the anchor assembly is implanted, wherein the rounded tip includes the radiopaque material and serves as the at least one fiducial marker.

8. The system of claim 1, wherein the connector is a wire made from or coated with the radiopaque material such that the connector serves as the at least one fiducial marker.

9. The system of claim 1, wherein the radiopaque material comprises gold, platinum, or tantalum.

10. The system of claim 1, wherein the at least one fiducial marker is configured to delineate the tissue for planning and execution of radiation therapy.

11. A method of delivering at least one fiducial marker to an interventional site within a body of a patient, comprising:
inserting an elongate member of a delivery device into the body of the patient;
advancing the elongate member to the interventional site;
deploying, via an actuator of the delivery device, an anchor assembly at the interventional site, the anchor assembly comprising a first anchoring component, a second anchoring component, a connector disposed between the first anchoring component and the second anchoring component, and at least one fiducial marker made of a radiopaque material incorporated onto at least a portion of the first anchoring component, the second anchoring component, the connector, or a combination thereof; and
positioning the first anchoring component and the second anchoring component to hold tissue captured therebetween in a compressed state via the connector such that the at least one fiducial marker delineates a boundary of the tissue.

12. The method of claim 11, wherein the interventional site comprises a gland or organ, and wherein the at least one fiducial marker delineates a boundary of the gland or organ for radiation therapy.

13. The method of claim 11, wherein the at least one fiducial marker is placed in intra-prostatic space, extra-prostatic space, on a prostatic capsule, or combinations thereof.

14. The method of claim 11, further comprising deploying a second anchor assembly at the interventional site, the second anchor assembly comprising a second fiducial marker, wherein the at least one fiducial marker and the second fiducial marker span the tissue.

15. The method of claim 11, wherein the at least one fiducial marker is positioned at a tissue plane.

16. A system for delivering and depositing at least one fiducial marker to tissue at an interventional site within a body of a patient, comprising:
an anchor assembly deployable at the interventional site, the anchor assembly comprising a first anchoring component, a second anchoring component, a connector disposed between the first anchoring component and the second anchoring component, and the at least one fiducial marker made of a radiopaque material, wherein:
the at least one fiducial marker comprises a radiopaque tag situated between the second anchoring component and the connector, and
the anchor assembly is configured to hold tissue captured between the first anchoring component and the second anchoring component in a compressed state via the connector such that the at least one fiducial marker delineates a boundary of the tissue; and
a delivery device carrying the anchor assembly, the delivery device comprising:
an elongate member configured to be inserted into the body of the patient and advanced to the interventional site; and
an actuator usable to deploy the anchor assembly at the interventional site.

17. The system of claim 16, further comprising a second anchor assembly, the second anchor assembly including a third anchoring component, a fourth anchoring component, a second connector, and at least one second fiducial marker, wherein the at least one fiducial marker and the at least one second fiducial marker are configured to delineate a boundary of a gland or organ for radiation therapy.

18. The system of claim 16, wherein the delivery device further comprises a needle advanceable from the elongate member, and wherein the needle is configured to deliver a dye or contrast agent to the interventional site.

19. The system of claim 16, wherein the first anchoring component comprises a first portion and a second portion, the first portion comprising a generally cylindrical structure and the second portion comprising a curved or angled leg extending from the first portion.

20. The system of claim 16, wherein the radiopaque material comprises gold, platinum, or tantalum.
